(19) United States
(12) Patent Application Publication
(10) Pub. No.: US 2014/0141987 A1
(43) Pub. Date: May 22, 2014

(54) METHOD FOR DIAGNOSING ARRHYTHMIA BASED ON SINGLE NUCLEOTIDE POLYMORPHISM IN CHROMOSOME 1Q24, NEURL GENE, OR CUX2 GENE

(71) Applicants: Kouichi Ozaki, Yokohama-shi (JP); Toshihiro Tanaka, Yokohama-shi (JP)

(72) Inventors: Kouichi Ozaki, Yokohama-shi (JP); Toshihiro Tanaka, Yokohama-shi (JP)

(73) Assignee: RIKEN, Saitama (JP)

(21) Appl. No.: 14/002,531

(22) PCT Filed: Mar. 14, 2012
(86) PCT No.: PCT/IB12/00475

§ 371 Date: Aug. 30, 2013

(30) Foreign Application Priority Data
Mar. 1, 2011 (JP) 2011-044157

(51) Int. Cl. C12Q 1/68

(52) U.S. Cl. CPC C12Q 1/6883
USPC 506/9; 435/6.11; 536/24.31; 536/24.33

(57) ABSTRACT

A method for diagnosing arrhythmia such as atrial fibrillation is provided. A single nucleotide polymorphism present in the region 24 of the long arm of the chromosome 1, NEURL gene, or CUX2 gene is analyzed, and the risk of developing arrhythmia and/or the presence or absence of the onset of arrhythmia is diagnosed on the basis of the analysis result.

### TECHNICAL FIELD

The present invention relates to a diagnosis method for determining the onset of arrhythmia such as atrial fibrillation and/or the risk of acquiring the same, and a reagent used in the diagnosis method.

### BACKGROUND ART

Arrhythmia refers to a symptom in which the heart rate or heartbeat rhythm is not constant. Arrhythmia is classified into various types based on the heart rate or the like. Of these, atrial fibrillation (AF) is a type of arrhythmia that is most frequently found in many countries including Japan. Atrial fibrillation refers to a symptom in which the number of pulse of the atria is irregular with high frequency and is accompanied by increase in morbidity and mortality. As risk factors of atrial fibrillation, gender, age, hypertension, obesity, and other heart diseases have been known. Thus, genetic factors related to these risk factors serve as determinants for predicting the risk of atrial fibrillation. Further, a family history of atrial fibrillation is thought to have genetic factors of its own. Thus, a positive family history of atrial fibrillation also serves as, separately from the above risk factors, determinants for predicting the risk of atrial fibrillation.

Familial studies of atrial fibrillation identified variations of several genes coding for ion channels in association with atrial fibrillation. Yet, those variations are not applicable to all cases of atrial fibrillation.

It has been recently attempted to identify genes and single nucleotide polymorphisms (SNPs) related to the onset of atrial fibrillation by genome-wide association study (GWAS). By GWAS employing Europeans and North Americans, it has been thus far suggested that genetic variations that are attributed to susceptibility to atrial fibrillation are present in several regions on the chromosome (Non-Patent Documents 1 to 5). In order to identify additional genetic variations associated with atrial fibrillation and to understand complicated genetic factors associated with atrial fibrillation, GWAS has to be carried out for Asians such as Japanese and for other race.

### PRIOR ART REFERENCES

### Non-Patent Documents

Non-Patent Document 1: Gudbjartsson D F. et. al., Nature. 2007 Jul. 19; 448(7151):353-357
Non-Patent Document 2: Gudbjartsson D F. et. al., Nat Genet. 2009 August; 41(8):876-878
Non-Patent Document 3: Benjamin E J. et. al., Nat Genet. 2009 August; 41(8):879-881
Non-Patent Document 4: Holm H. et. al., Nat Genet. 2010 February; 42(2):117-122
Non-Patent Document 5: Ellinor P T. et. al., Nat Genet. 2010 March; 42(3):240-244

### SUMMARY OF THE INVENTION

### Problems to be Solved by the Invention

An object of the present invention is to provide a method for accurately diagnosing the risk of developing arrhythmia such as atrial fibrillation and/or the onset of the same, and a diagnosis reagent used in the method.

### Means for Solving the Problems

The present inventors have intensively studied in order to solve the above object to identify that a single nucleotide polymorphism (SNP) present in the region 24 of the long arm of the chromosome 1 (1q24), NEURL gene, or CUX2 gene is associated with atrial fibrillation. And, they found that the risk of developing arrhythmia such as atrial fibrillation or the onset of the same can be accurately estimated by analyzing these polymorphisms, thereby completing the present invention.

Accordingly, the present invention is as follows:

[1] A method for diagnosing the onset of arrhythmia and/or the risk of acquiring arrhythmia comprising:

analyzing a single nucleotide polymorphism present in any of the following regions (1) to (3) and;

diagnosing arrhythmia on the basis of the analysis result:

(1) the region 24 of the long arm of the chromosome 1;

(2) NEURL gene;

(3) CUX2 gene.

[2] The method according to [1], wherein said single nucleotide polymorphism is a polymorphism of a nucleotide corresponding to the nucleotide at position 61 in a nucleotide sequence of SEQ ID NO: 1, 2, or 3, or a polymorphism of a nucleotide showing linkage disequilibrium with said nucleotide.
[3] The method according to [2], wherein said nucleotide showing linkage disequilibrium is a nucleotide corresponding to the nucleotide at position 61 in a nucleotide sequence selected from SEQ ID NOS: 4 to 20.
[4] The method according to any one of [1] to [3], wherein said arrhythmia is atrial fibrillation.
[5] A probe for diagnosing arrhythmia, wherein said probe has a sequence of 10 nucleotides or more containing the nucleotide at position 61 in a nucleotide sequence selected from SEQ ID NOS: 1 to 20, or has a complementary sequence thereof.
[6] A primer for diagnosing arrhythmia, wherein said primer can amplify a region comprising the nucleotide at position 61 in a nucleotide sequence selected from SEQ ID NOS: 1 to 20.

### Effect of the Invention

According to the present invention, the risk of developing (risk of acquiring) arrhythmia which has been thus far difficult to be predicted can be accurately and simply predicted. Further, the onset of arrhythmia can be accurately and simply diagnosed. That is, the present invention contributes to prophylaxis and early treatment of arrhythmia.

### BRIEF DESCRIPTION OF THE DRAWINGS

FIG. 1 is a figure showing linkage disequilibrium (LD) maps and genes in the region 24 of the long arm of the human chromosome 1 (1q24 region). The down-pointing arrow indicates the position of rs639652. The upper row shows an LD map based on Japanese database (JPT) of the HapMap project whereas the lower row shows an LD map based on European database (CEU).

### MODE FOR CARRYING OUT THE INVENTION

### <1> Method of the Present Invention

The method of the present invention is a method for diagnosing the onset of arrhythmia and/or the risk of acquiring arrhythmia comprising: analyzing an SNP present in the region 24 of the long arm of the human chromosome 1, NEURL gene, or CUX2 gene; and diagnosing arrhythmia on the basis of the analysis result. Note that, the term “diagnosis/diagnosing” in the present invention includes a diagnosis of the risk of developing arrhythmia and a diagnosis of the presence or absence of onset of arrhythmia. In the method of the present invention, the analysis result of an SNP is associated with the risk of developing arrhythmia and/or the presence or absence of the onset of arrhythmia.

Examples of arrhythmia include tachyarrhythmia, bradyarrhythmia, and premature contraction. Examples of tachyarrhythmia include sinus tachycardia, ventricular tachycardia, atrial fibrillation, atrial flutter, multifocal atrial tachycardia, ventricular fibrillation, ventricular flutter, and supraventricular tachycardia. Examples of bradyarrhythmia include sinoatrial block, atrioventricular block, junctional rhythm, sick sinus syndrome, respiratory arrhythmia, and bundle branch block. Examples of premature contraction include premature atrial contraction, and premature ventricular contraction. Of these, it is preferred to diagnose atrial fibrillation.

Specific examples of the SNPs present the region 24 of the long arm of the chromosome 1 (1q24 region) in human include human rs639652. Here, the rs number represents a registration number of the dbSNP database National Center for Biotechnology Information (http//www.ncbi.nlm.nih.gov/projects/SNP/). rs639652 means a polymorphism of cytosine(C)/thymine(T) in the nucleotide at position 21103228 of GenBank Accession No. NT_{—}004487.18. Although the risk allele is C, because the relationship between this SNP and arrhythmia is a recessive model, only in the case of CC, which is homozygous, the possibility of arrhythmia or the risk of developing arrhythmia increases.

Further, PRRX1 gene is present adjacent to rs639652 in the chromosome 1q24 region. PRRX1 gene codes for a homeobox protein belonging to the pair type family and is involved in expression of various genes and generation of muscles. rs639652 is located in the promoter region of PRRX1 gene and it is thus thought to be involved in expression of PRRX1 gene. Therefore, it is also possible to diagnose arrhythmia by analyzing an SNP present in PRRX1 gene. Specific examples of PRRX1 gene include the region of 170633313 to 170708541 of GenBank Accession No. NC_{—}000001.10.

NEURL gene is present in the region 25.1 of the long arm of the chromosome 10 (10q25.1 region) in human. Specific examples of NEURL gene include the region of 105253735 to 105352309 of GenBank Accession No. NC_{—}000010.10.

Specific examples of the SNPs present in NEURL gene include human rs6584555. rs6584555 means a polymorphism of cytosine(C)/thymine(T) in the nucleotide at position 24048137 of GenBank Accession No. NT_{—}030059.12 and in cases where this nucleotide is C, the possibility of arrhythmia or the risk of developing arrhythmia is high. Further, when the analysis is carried out by taking alleles into consideration, the possibility of arrhythmia or the risk of developing arrhythmia is higher in the order of rs6584555 being CC>CT>TT from the highest to the lowest.

CUX2 gene codes for a cut-like homeobox 2 protein and present in the 24 region of the long arm of the chromosome 12 (12q24 region) in human. Specific examples of CUX2 gene include the region of 111471828 to 111788358 of GenBank Accession No. NC_{—}000012.11.

Specific examples of the SNPs present in CUX2 gene include human rs6490029. rs6490029 means a polymorphism of adenine(A)/guanine(G) in the nucleotide at position 2267966 of GenBank Accession No. NT_{—}009775.16. Although the risk allele is A, because the relationship between this SNP and arrhythmia is a recessive model, only in the case of AA, which is homozygous, the possibility of arrhythmia or the risk of developing arrhythmia increases.

With regard to rs639652, rs6584555, and rs6490029, the sequences of a total length of 121 bp, each of which comprises the SNP nucleotide and the 60 bp regions upstream and downstream thereof, are shown in SEQ ID NOS: 1, 2, and 3, respectively. The 61th nucleotide has a polymorphism.

In the present invention, a nucleotide corresponding to the above-described nucleotide is analyzed. The “nucleotide corresponding to the above-described nucleotide” refers to the corresponding nucleotide in the above-described region. That is, the expression “a nucleotide corresponding to the above-described nucleotide is analyzed” includes a case of analyzing the corresponding nucleotide in the above-described region even if the above-described sequence slightly changes at a position other than the SNP position due to a racial difference or the like.

Additionally, the nucleotide to be analyzed in the present invention is not limited to the above-described nucleotide, and a polymorphism of a nucleotide showing linkage disequilibrium with the above-described nucleotide can be analyzed. Herein, the “nucleotide showing linkage disequilibrium with the above-described nucleotide” refers to a nucleotide that satisfies a relationship of r²>0.5, preferably r²>0.8, or more preferably r²>0.9 with the above-described nucleotide. In addition, the nucleotide showing linkage disequilibrium with the above-described nucleotide can be identified, for example, by using the HapMap database (http://www.hapmap.org/index.html.ja) or the like. Alternatively, the nucleotide showing linkage disequilibrium with the above-described nucleotide can be identified by analyzing the sequences of DNAs extracted from a plurality of persons (usually, about 20 to 40 persons) and then screening a SNP showing linkage disequilibrium.

Examples of the nucleotides showing linkage disequilibrium with rs639652 with r²>0.8 include rs12760630, rs736791, rs541557, rs577676, rs763567, rs6658866, rs3903239, rs6677540, rs2022372, rs1234275, rs12755237, and rs593560. The sequences of a total length of 121 bp, each of which comprises the SNP nucleotide and the 60 bp regions upstream and downstream thereof, are shown in SEQ ID NOS: 4 to 15, respectively. The 61th nucleotide has a polymorphism. Because the relationship between these SNPs and arrhythmia is a recessive model, only in the case of being homozygous for the risk allele, the possibility or risk of developing arrhythmia increases.

Examples of the nucleotides showing linkage disequilibrium with rs6584555 with r²>0.8 include rs7904046, rs6584554, rs6584557, and rs7069733. The sequences of a total length of 121 bp, each of which comprises the SNP nucleotide and the 60 bp regions upstream and downstream thereof, are shown in SEQ ID NOS: 16 to 19, respectively. The 61th nucleotide has a polymorphism. With regard to these SNP, the possibility of arrhythmia or the risk of developing arrhythmia increases in the order of homozygotes for the risk allele>heterozygotes for the risk allele and non-risk allele>homozygotes for the non-risk allele.

Examples of the nucleotides showing linkage disequilibrium with rs6490029 with r²>0.8 include rs916682. The sequence of a total length of 121 bp comprising this SNP nucleotide and the 60 bp regions upstream and downstream thereof is shown in SEQ ID NO: 20. The 61th nucleotide has a polymorphism. Because the relationship between this SNP and arrhythmia is a recessive model, only in the case of being homozygous for the risk allele, the possibility of arrhythmia or the risk of developing arrhythmia increases.

With regard to each of the nucleotides showing linkage disequilibrium with rs639652, rs6584555, or rs6490029 with r²>0.8, the combination of the alleles and the risk allele are shown in Table 1.

TABLE 1
SNPs showing linkage disequilibrium with r² > 0.8

| marker SNP | SNP showing linkage disequilibrium | alleles | risk allele |
|---|---|---|---|
| rs639652 | rs541557 | A/G | A |
| | rs577676 | C/T | C |
| | rs593560 | A/G | G |
| | rs736791 | A/G | A |
| | rs763567 | A/G | G |
| | rs1234275 | A/G | A |
| | rs2022372 | A/T | A |
| | rs3903239 | A/G | G |
| | rs6658866 | A/G | G |
| | rs6677540 | A/G | A |
| | rs12755237 | A/G | A |
| | rs12760630 | A/G | A |
| rs6584555 | rs6584554 | A/G | A |
| | rs6584557 | A/G | G |
| | rs7069733 | C/G | G |
| | rs7904046 | A/C | A |
| rs6490029 | rs916682 | A/G | G |

Arrhythmia can be diagnosed by analyzing the type of the nucleotide of the above-described SNP and relating the obtained result to arrhythmia on the basis of the criteria as described above. One of the above-described SNPs can be analyzed solely or a plurality of SNPs including at least one of the above-described SNPs can be collectively analyzed (Haplotype Analysis). For example, a plurality of the above-described SNPs can be collectively analyzed, or at least one of the above-described SNPs can be analyzed in combination with known SNPs associated with arrhythmia (for example, Non-Patent Literatures 1 to 5) or SNPs showing linkage disequilibrium with the known SNPs. By collectively analyzing a plurality of SNPs associated with arrhythmia, the accuracy of diagnosis of arrhythmia can be improved. For any SNP, either strand of double-stranded DNA can be analyzed. For example, regarding the sequence of the PRRX1 gene, NEURL gene, or CUX2 gene, either the sense strand or antisense strand of the gene can be analyzed.

The sample used for the SNP analysis is not particularly limited as long as it is a sample containing the chromosomal DNA, and examples of such a sample include body fluids such as blood and urine, cells such as oral mucous membrane, and body hair such as hair on the head. These samples can be directly used for the SNP analysis. Yet, it is preferred that the chromosomal DNA is isolated from the samples by a conventional method and the isolated chromosomal DNA is used for the analysis.

The SNP analysis can be carried out by a usual method for analyzing gene polymorphism. Examples of such a method include, but not limited to, sequencing analysis, PCR, hybridization, and invader assay.

Sequencing analysis can be carried out by a usual method. Specifically, sequencing reaction is performed using primers to be located at a position of several ten nucleotides on the 5′ side from a polymorphic nucleotide, and the type of the nucleotide at the corresponding position can be determined on the basis of the result of the analysis. In addition, it is preferred that before sequencing reaction, a fragment containing the SNP site is preliminarily amplified by PCR or the like.

Also, the SNP analysis can be carried out by investigating the presence or absence of amplification by PCR. For example, primers which have sequences corresponding to a region containing a polymorphic nucleotide and whose 3′ ends correspond to the respective polymorphisms are prepared. PCR is performed using each primer, and the type of polymorphism can be determined on the basis of the presence of absence of an amplification product. Furthermore, the presence or absence of amplification can be detected by the LAMP method (Japanese Patent No. 3313358), the NASBA method (Nucleic Acid Sequence-Based Amplification; Japanese Patent No. 2843586), the ICAN method (Japanese Patent Application Laid-Open Publication No. 2002-233379), or the like. Other than these, a single-strand amplification method can also be used.

In addition, a DNA fragment containing a SNP site is amplified, and the type of polymorphism can be determined on the basis of a difference in electrophoretic mobility of amplification product. An example of such a method includes a PCR-SSCP (single-strand conformation polymorphism) method (Genomics. 1992 Jan. 1; 12(1): 139-146.). Specifically, at first, DNA containing a target SNP is amplified and the amplified DNA is dissociated into single-stranded DNAs. Next, the dissociated single-stranded DNAs are separated on a non-denaturing gel, and the type of polymorphism can be determined on the basis of the mobility difference between the separated single-stranded DNAs on the gel.

Furthermore, when a polymorphic nucleotide is contained in a restriction enzyme recognition sequence, analysis can be carried out on the basis of the presence or absence of cleavage by the restriction enzyme (RFLP method). In this case, at first, a DNA sample is cleaved by a restriction enzyme. Next, DNA fragment(s) are separated and the type of polymorphism can be determined on the basis of the size of the detected DNA fragment(s).

It is also possible to analyze the type of polymorphism by detecting the presence or absence of hybridization. Specifically, by preparing probes corresponding to the respective nucleotides and investigating which probe hybridizes to the DNA, the type of nucleotide of the SNP can be determined.

By determining the type of the nucleotide of an SNP, data for diagnosing arrhythmia can be obtained.

### <2> Diagnosis Reagent of Present Invention

The present invention also provides a diagnosis reagent, such as a primer or a probe, for diagnosing arrhythmia. An example of such a probe includes a probe that contains the above-described SNP site and allows for the determination of the type of the nucleotide at the SNP site on the basis of the presence or absence of hybridization. Specific examples of the probe include a probe with a length of 10 or more nucleotides that has a sequence comprising the 61st nucleotide in a nucleotide sequence selected from SEQ ID NOS: 1 to 20 or has a complementary sequence thereof. The length of the probe is preferably 15 to 35 nucleotides, or more preferably 20 to 35 nucleotides.

As well, examples of the primer include a primer usable in PCR for amplifying the above-described SNP site and a primer usable for sequencing analysis (sequencing) of the above-described SNP site. Specific examples of the primer include a primer capable of amplifying or sequencing a region comprising the 61st nucleotide in a nucleotide sequence selected from SEQ ID NOS: 1 to 20. The length of primer is preferably 10 to 50 nucleotides, more preferably 15 to 35 nucleotides, or further preferably 20 to 35 nucleotides.

Examples of the primer for sequencing or amplifying the above-described SNP site include a primer having a sequence of the 5′ side region of the above-described nucleotide, preferably a sequence of 30 to 100 nucleotides upstream of the above-described nucleotide, and a primer having a complementary sequence of the 3′ side region of the above-described nucleotide, preferably a complementary sequence of a region of 30 to 100 nucleotides downstream of the above-described nucleotide. Examples of the primer used to determine a polymorphism on the basis of the presence or absence of amplification by PCR include a primer that has a sequence comprising the above-described nucleotide and comprises the above-described nucleotide on the 3′ side of the primer, and a primer that has a sequence complementary to the sequence comprising the above-described nucleotide and comprises the nucleotide complementary to the above-described nucleotide on the 3′ side of the primer.

The diagnosis reagent of the present invention can include, in addition to the primer(s) and probe(s), polymerase and buffer for PCR, reagents for hybridization, and/or the like.

### EXAMPLES

By way of examples, the present invention will be further concretely described below. However, the present invention is by no means limited thereto.

(1) Identification of SNPs Associated with Atrial Fibrillation

In order to identify genetic variations determining the susceptibility to atrial fibrillation, a genome-wide association study (GWAS) was carried out with Japanese subjects. GWAS is a genetic statistical method for screening genetic variations associated with phenotypes such as diseases. For example, genetic variations associated with a certain disease can be found by using SNPs at several hundred thousand to one million sites covering the whole human genome and statistically testing whether there is any difference in polymorphism frequencies between patients with the disease (cases) and subjects without the disease (controls).

### <Subject>

All of the subjects with atrial fibrillation (case) who were employed in the primary test of GWAS and most of the subjects with atrial fibrillation who were employed in the secondary test were employed, through clinical diagnosis, from a group of patients with atrial fibrillation, registered in BioBank Japan (BBJ), Institute of Medical Science, The University of Tokyo (Nakamura, Y. The BioBank Japan Project. Clin Adv Hematol Oncol 5, 696-7 (2007)). A part of the subjects with atrial fibrillation who were employed in the secondary test were obtained from Department of Cardiovascular Medicine, Tokyo Medical and Dental University. All of the subjects with atrial fibrillation were diagnosed as atrial fibrillation by the standard 12-lead electrocardiogram (ECG).

As control subjects (control) for the primary test of GWAS, 2,444 patients with a disease other than atrial fibrillation who were resistered in BBJ and 906 healthy volunteers who were recruited from Osaka-Midousuji Rotary Club were employed. As control subjects for the secondary test, 17,190 patients with a disease other than atrial fibrillation who were resistered in BBJ were employed.

It was confirmed by major component analysis (PCA) that the subjects did not show any population stratification. That is, there are considered to be no genetic differences other than genetic factor associated with atrial fibrillation between the subjects with atrial fibrillation and control subjects.

The present study was approved by the Ethical Committee in Institute of Medical Science, The University of Tokyo and the Institute of Physical and Chemical Research, Yokohama and informed consent was obtained from all of the participants or from, in cases where participants are younger than 20 years old, their parents.

### <Statistical Analysis>

In the primary test and secondary test of GWAS, association between each of the SNPs on the autosomal chromosome and atrial fibrillation was evaluated by Cochran-Armitage trend test. A statistical analysis in which the primary test and secondary test were combined was carried out by Mantel-Haenszel method.

### <Primary Test of GWAS>

The genotypes of 843 subjects with atrial fibrillation were analyzed using Human610-Quad BeadChip (Illumina, Inc.). The genotypes of 3,350 control subjects were analyzed using HumanHap550v3 Genotyping BeadChip (Illumina, Inc.). An association analysis was carried out for about 430,000 SNPs on the autosomal chromosome that satisfy a minor allele frequency (MAFs) of >0.01 in the control subjects.

As a result of GWAS, it was confirmed that rs1906599, which is located adjacent to PITX2 gene in the 25 region of the long arm of the chromosome 4, satisfied P<1.0×10⁷ which was set as a threshold value for genome-wide significance, and thus, was significantly associated with atrial fibrillation (Table 1). It has been already reported that the region where rs1906599 is present is associated with atrial fibrillation in Europeans and North Americans.

TCBLE 2
SNPs associated with atrial fibrillation

| dbSNP ID | | | | | | | | | | |
|---|---|---|---|---|---|---|---|---|---|---|
| Chromosome | | AF | | | | | | | CO | |
| Gene name | Sample | 11 | % | 12 | % | 22 | % | SUM | 11 | % |
| rs639652 | GWAS_L10 | 288 | 34.2 | 401 | 47.6 | 154 | 18.3 | 843 | 957 | 28.6 |
| 1q | sstage2 | 585 | 35.4 | 776 | 46.9 | 293 | 17.7 | 1554 | 5068 | 29.5 |
| PRRX1 | Total | 873 | 35.0 | 1177 | 47.1 | 447 | 17.9 | 2497 | 6025 | 29.3 |
| rs1906599 | GWAS_L10 | 527 | 62.5 | 270 | 32.0 | 46 | 5.5 | 843 | 1536 | 45.9 |
| 4q | stage2 | 920 | 65.9 | 425 | 30.5 | 50 | 3.6 | 1395 | 7891 | 45.9 |
| PITX2 | Total | 1447 | 84.7 | 695 | 31.1 | 98 | 4.3 | 2238 | 9427 | 45.9 |
| rs6466579 | GWAS_L10 | 490 | 58.1 | 311 | 36.9 | 42 | 5.0 | 843 | 1767 | 52.7 |
| 7q | stage2 | 978 | 59.3 | 587 | 35.6 | 84 | 5.1 | 1649 | 9207 | 53.6 |
| CAV1, 2 | Total | 1468 | 58.9 | 898 | 36.0 | 126 | 5.1 | 2492 | 10974 | 53.4 |
| rs6584555 | GWAS_L10 | 609 | 72.2 | 220 | 26.1 | 14 | 1.7 | 843 | 2623 | 78.3 |
| 10q | sstage2 | 1138 | 70.6 | 446 | 27.6 | 30 | 1.9 | 1814 | 13303 | 77.4 |
| NEURL | Total | 1747 | 71.1 | 666 | 27.1 | 44 | 1.8 | 2457 | 15926 | 77.5 |
| rs6490029 | GWAS_L10 | 394 | 46.8 | 374 | 44.4 | 74 | 8.8 | 842 | 1397 | 41.7 |
| 12q | sstage2 | 741 | 45.3 | 753 | 46.0 | 142 | 8.7 | 1838 | 7160 | 41.7 |
| CUX2 | Total | 1135 | 45.8 | 1127 | 45.5 | 216 | 8.7 | 2478 | 8557 | 41.7 |
| rs12932445 | GWAS_L10 | 289 | 34.3 | 391 | 46.4 | 163 | 19.3 | 843 | 1320 | 39.4 |
| 16q | stage2 | 443 | 31.9 | 650 | 46.8 | 296 | 21.3 | 1389 | 6967 | 40.5 |
| ZFHX3 | Total | 732 | 32.8 | 1041 | 46.6 | 459 | 20.6 | 2232 | 8287 | 40.3 |

| dbSNP ID | | | | | | | | |
|---|---|---|---|---|---|---|---|---|
| Chromosome | | CO | | | | | | |
| Gene name | Sample | 12 | % | 22 | % | SUM | OR | P |
| rs639652 | GWAS_L10 | 1857 | 49.5 | 738 | 22.0 | 3350 | 1.21 | 6.1E−04 |
| 1q | sstage2 | 8511 | 49.5 | 3610 | 21.0 | 17189 | 1.21 | 4.2E−07 |
| PRRX1 | Total | 10168 | 49.5 | 4348 | 21.2 | 20639 | 1.21 | 1.1E−09 |
| rs1906599 | GWAS_L10 | 1464 | 43.7 | 350 | 10.4 | 3350 | 1.75 | 9.0E−18 |
| 4q | stage2 | 7496 | 43.6 | 1802 | 10.5 | 17189 | 2.06 | 6.7E−49 |
| PITX2 | Total | 8960 | 43.6 | 2152 | 10.5 | 20539 | 1.93 | 3.9E−65 |
| rs6466579 | GWAS_L10 | 1324 | 39.5 | 259 | 7.7 | 3350 | 1.24 | 7.6E−04 |
| 7q | stage2 | 8766 | 39.4 | 1215 | 7.1 | 17188 | 1.23 | 1.5E−06 |
| CAV1, 2 | Total | 8090 | 39.4 | 1474 | 7.2 | 20538 | 1.23 | 5.0E−09 |
| rs6584555 | GWAS_L10 | 884 | 20.4 | 43 | 1.3 | 3350 | 1.33 | 2.8E−04 |
| 10q | sstage2 | 3617 | 21.0 | 270 | 1.6 | 17190 | 1.36 | 3.8E−09 |
| NEURL | Total | 4301 | 20.9 | 313 | 1.5 | 20540 | 1.34 | 5.3E−12 |
| rs6490029 | GWAS_L10 | 1533 | 45.8 | 420 | 12.6 | 3360 | 1.22 | 6.3E−04 |
| 12q | sstage2 | 7831 | 45.6 | 2198 | 12.8 | 17189 | 1.19 | 9.4E−06 |
| CUX2 | Total | 9364 | 45.8 | 2618 | 12.7 | 20539 | 1.2 | 2.6E−08 |
| rs12932445 | GWAS_L10 | 1581 | 47.2 | 449 | 13.4 | 3350 | 1.26 | 3.1E−05 |
| 16q | stage2 | 7826 | 45.5 | 2397 | 13.9 | 17190 | 1.39 | 1.2E−16 |
| ZFHX3 | Total | 9407 | 45.8 | 2846 | 13.9 | 20540 | 1.35 | 9.0E−21 |

“AF” represents the subjects with atrial fibrillation and “CO” represents the control subjects.
“1” represents the major allele and “2” represents the minor allele. That is, “11” represents the homozygote of major allele; “12” represents the heterozygote of major allele and minor allele; and “22” represents the homozygote of minor allele.
“OR” represents an odds ratio (OR) of the alleles at a confidence interval (CI) of 95%.

### <Secondary Test of GWAS>

In order to validate the result of the primary test, the secondary test was carried out by employing the 1,600 subjects with atrial fibrillation and 17,190 control subjects. The genotypes of subjects with prostate cancer were analyzed by a multiplex-PCR invader assay (Third Wave Technologies) (Ohnishi, Y. et al. J Hum Genet 46, 471-7 (2001)). The genotypes of control subjects were analyzed using Human610-Quad BeadChip (Illumina, Inc.). Note that all of the subjects are independent of the subjects employed in GWAS.

As SNPs used in the secondary test, top 500 SNPs with the lowest p values in the primary test of GWAS were selected. The linkage disequilibrium (LD) coefficient (r²) was calculated for each pair of these 500 SNPs; and 150 SNPs which are highly linked to other SNPs with r²>0.8 were excluded and the remaining 350 SNPs were subjected to the secondary test.

As a result of meta-analysis of the primary test and secondary test, it was found that SNPs at additional 5 sites satisfied P<1.0×10⁻⁷ which was set as a threshold value for genome-wide significance, and thus, was significantly associated with atrial fibrillation (Table 1). SNPs at these 5 sites are rs639659 in the region 24 of the long arm of the chromosome 1 (1q24 region), rs6584555 in the 25.1 region of the long arm of the chromosome 10 (10q25.1 region), rs6490029 in the region 24 of the long arm of the chromosome 12 (12q24 region), rs6466579 in the region 31 of the long arm of the chromosome 7 (7q31 region), and rs12932445 in the region 22 of the long arm of the chromosome 16 (16q22 region).

Of these, CAV1/CA2 gene in which rs6466579 is located and ZFHX3 gene in which rs12932445 is located have been already reported to be associated with atrial fibrillation in Europeans and North Americans.

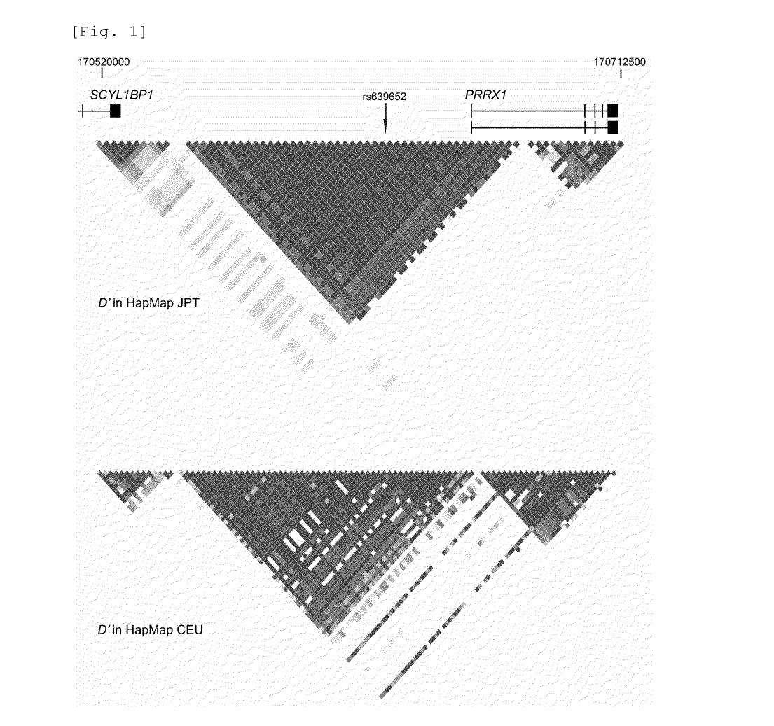

rs639652 (P=1.1×10⁻⁹) is located in the 1q24 region of the chromosome and, to be specific, is located in a promoter region of PRRX1 gene. Therefore, it is thought that this SNP is related to expression of PRRX1 gene. Linkage disequilibrium (LD) maps of the region of about 200 kb adjacent to rs639652 are shown in FIG. 1. The LD map was prepared based on each of Japanese database (JPT) and European database (CEU) in the HapMap project using Haploview Software (Barrett, J. et al. Bioinformaties 21, 263. 265 (2005)).

PRRX1 gene codes for a homeobox protein belonging to the pair type family. Prrx1 protein is a co-activator and improves DNA binding ability of serum response factor (Grueneberg D A. et. al., Science. 1992 Aug. 21; 257(5073):1089-95). The serum response factor is a factor required for expression induction of various genes by a growth factor or differentiation factor. Thus, PRRX1 gene is involved in expression induction of various genes. In addition, Prrx1 protein regulates creatine kinase in the muscle, and thus, is thought to contribute to development of various types of muscles in the mesoderm (Cserjesi P. et. al., J Biol Chem. 1994 Jun. 17; 269(24):16740-5).

PITX2 gene, which has been already reported to be associated with atrial fibrillation in Europeans and North Americans, codes for a transcription factor belonging to the RIEG/PITX homeobox family, and ZFH3 gene codes for a transcription factor having a zinc finger domain and homeodomain (Non-Patent Documents 1 to 3). At the anastomotic site of the pulmonary vein and left atrium, the myocardial muscles develop while sleeving the pulmonary vein and the myocardial muscles at this site are known to cause atrial fibrillation. It has been recently suggested in Pitx2-deficient mice that initial formation of pulmonary myocardial cells does not take place and pulmonary myocardial muscles does not developed into the pulmonary vein (Mommersteeg M T. et. al., Circ Res. 2007 Oct. 26; 101(9):902-9). Further, ZFH3 gene is necessary for transcription activation of POU1F1 gene, and the transcription factor coded by POU1F1 gene interacts with Pitx2 protein thereby to promote DNA binding ability and transcriptional activity of Pitx2 protein (Amendt B A. et. al., J Biol Chem. 1998 Aug. 7; 273(32):20066-72).

Prrx1 protein resembles Pitx2 protein and Zfh3 protein as a transcription factor; and it is thought that PRRX1 gene, similarly to PITX2 gene and ZFH3 gene, is associated with atrial fibrillation.

rs6584555 (P=5.3×10⁻¹²) is located in the 10q25.1 region of the chromosome and specifically located in NEURL gene.

rs6490029 (P=2.6×10⁻⁸) is located in the 12q24 region of the chromosome and specifically located in CUX2 gene. CUX2 gene codes for a cut-like homeobox 2 protein. Cux2 protein is thought to function as a homeobox transcription factor.

Further, no significant gene interaction was recognized among any genes in which these SNPs are located.

As described above, three SNPs associated with atrial fibrillation were newly identified. These SNPs are useful in the diagnosis of arrhythmia such as atrial fibrillation.

1. A method for diagnosing the onset of arrhythmia and/or the risk of acquiring arrhythmia comprising:
analyzing a single nucleotide polymorphism present in any of the following regions (1) to (3) and;
diagnosing arrhythmia on the basis of the analysis result:
(1) the region 24 of the long arm of the chromosome 1;
(2) NEURL gene;
(3) CUX2 gene.

2. The method according to claim 1, wherein said single nucleotide polymorphism is a polymorphism of a nucleotide corresponding to the nucleotide at position 61 in a nucleotide sequence of SEQ ID NO: 1, 2, or 3, or a polymorphism of a nucleotide showing linkage disequilibrium with said nucleotide.

3. The method according to claim 2, wherein said nucleotide showing linkage disequilibrium is a nucleotide corresponding to the nucleotide at position 61 in a nucleotide sequence selected from SEQ ID NOS: 4 to 20.

4. The method according to claim 1, wherein said arrhythmia is atrial fibrillation.

5. A probe for diagnosing arrhythmia, wherein said probe has a sequence of 10 nucleotides or more containing the nucleotide at position 61 in a nucleotide sequence selected from SEQ ID NOS: 1 to 20, or has a complementary sequence thereof.

6. A primer for diagnosing arrhythmia, wherein said primer can amplify a region comprising the nucleotide at position 61 in a nucleotide sequence selected from SEQ ID NOS: 1 to 20.


SEQUENCE LISTING (20 sequences)

SEQ ID NO: 1 - DNA, 121 bp, Homo sapiens
ttaagtgcaaaatgaaaaacaaatgtgtaaggcatacaaaagaagactaagaaagcactaytgaaggacataaaagaagatatgacttgacatggatgggcatttcctacccagttgaaaa

SEQ ID NO: 2 - DNA, 121 bp, Homo sapiens
gagatgcaaattagaattgttgggtggactttgaataaggctcctttaaagaggggcaggyagctaatatatgtcctttggttttggttttctgtgttctttctgtttcttgcctggaatg

SEQ ID NO: 3 - DNA, 121 bp, Homo sapiens
gaccggtggctgccttattgaacagctccgctccagtggggaaaacttaagaccaaaactrtcatcctccttataatgttagatttagtcttccccactggaagagataaatcatggacca

SEQ ID NO: 4 - DNA, 121 bp, Homo sapiens
agattttttattcctataaatatttttgagtcttattttaggacattgttaaattatttaraaacagtttaacccttgagctttattttaggacattgttaaattattattattattgtta

SEQ ID NO: 5 - DNA, 121 bp, Homo sapiens
cttaattgttgattgggctctaatggtctgttcactgtactactgcctatatgaacatgarccctattcctaattacaacccatttttgttgttgttatctagatgacatcaagatgtgtg

SEQ ID NO: 6 - DNA, 121 bp, Homo sapiens
tctcattttggtttttctttcctttcgttttatattgaagtaatttagattgggtttaatyccaagtagtttttgcagcaataaagtctattttgctaaagacttccatgttccatgtcta

SEQ ID NO: 7 - DNA, 121 bp, Homo sapiens
gaacaaggagactcacccaagtcttcctgattccaaagcatgtgctctttccattacacayggaatgttgggaattcagattatatttcgttttggcctaggttagagacctgctctggac

SEQ ID NO: 8 - DNA, 121 bp, Homo sapiens
tggtctcgaactcctctttcagcactttgaatatgacatttcactgccttcaggcctccartgtttctactgagaaatgagtcttcactcgtattgacgctgctctgtacataatgagtca

SEQ ID NO: 9 - DNA, 121 bp, Homo sapiens
ctggggtttatcaaaagtcaaacgtagctttaatcacatcaacatgctaattacaggcaarcaataaaagctttaagaaatgattgtgtgggttttattactattgacagtcactgaggta

SEQ ID NO: 10 - DNA, 121 bp, Homo sapiens
aattaaacttctaaagatgaaaacaatgacccaaatgaaaaatacaatggacaggattaayggcagattaaacattgcagaggaaaagattggtagtgaacttaaagatacagcaatagaa

SEQ ID NO: 11 - DNA, 121 bp, Homo sapiens
ggtggctttgtttacactattaggggaaaaccacctactcaagcctcagtaatgatggacraccctccccccaccaagcttgagcatcccaggtcaatttcagattgctgtgctggcagcg

SEQ ID NO: 12 - DNA, 121 bp, Homo sapiens
gatatgtgttggctgattatatagccacgtggaccatacaagaacacagagaacccctggwgagtctcatgaaggaggtaccatccttacctgaattaatataacacttttgcacagggta

SEQ ID NO: 13 - DNA, 121 bp, Homo sapiens
accaccagtctagaattcttcattcttctaaactaaaagagaaagattttctccaacaagyaaaaactgaggaatttgtcactagcagatctgccttgcaggaaatgttaaaagaattttt

SEQ ID NO: 14 - DNA, 121 bp, Homo sapiens
atttgtttagctttttggatctgtaagcttatagttttcattcaatttggaactgttttgrtcattattttggcatcacagtgtccctccatatctgctggttctacatctaagtgttcaa

SEQ ID NO: 15 - DNA, 121 bp, Homo sapiens
aggggtctgagatgctgttaattacttaatttaggcttttcctttggccctgatggatgargggctaggataaatagatgttataaaaatttttaaaaatacttctcttaagatttgagaa

SEQ ID NO: 16 - DNA, 121 bp, Homo sapiens
atatattttaaaagcaatgttatttctgttttttattactagcaaccaaatgcaattcctmacagacacagctggttaacatagcagtgcatcaggaggaagtcagctgtattgcatatat

SEQ ID NO: 17 - DNA, 121 bp, Homo sapiens
ccagaggaaatggaactcatcttatggctgaggaccctgtttttaacttgagcgtgcaatrtgtccaatgaaaagaccacacatttcagcttcttgtccagcttggtgtaggcatgtgact

SEQ ID NO: 18 - DNA, 121 bp, Homo sapiens
tatgttaaagaaaacacttaatatattttaaaagcaatgttatttctgttttttattactrgcaaccaaatgcaattcctcacagacacagctggttaacatagcagtgcatcaggaggaa

SEQ ID NO: 19 - DNA, 121 bp, Homo sapiens
actatagattagtttacagtttttagagttctatgtaatgggaatgatacagtatggtctstttttttctctagcttctttcactcagcataattagtttgagattcatgcatgctatggt

SEQ ID NO: 20 - DNA, 121 bp, Homo sapiens
gtgtccgtggagacagactctcatgaatagtaaaagcttctggcacacagtaaacggtcgrtgccatttgtttttattagcccatcagggatgccccccgcccccgccccaagattctgaa